(19) United States
(12) Patent Application Publication
(10) Pub. No.: US 2011/0245875 A1
(43) Pub. Date: Oct. 6, 2011

(54) SUBLAMINAR WIRED SCREWED DEVICE FOR SPINAL FUSION

(75) Inventor: Aftab S. Karim, Flint, MI (US)

(73) Assignee: Neurosurj Research & Development, LLC

(21) Appl. No.: 12/754,448

(22) Filed: Apr. 5, 2010

(51) Int. Cl. A61B 17/70; A61B 17/86; A61B 17/88

(52) U.S. Cl. 606/263; 606/246; 606/264; 606/305; 606/279

(57) ABSTRACT

The invention relates to an anchoring device to be used for stabilizing the spine and the methods pertaining thereto.

### FIELD OF THE INVENTION

The invention relates to an anchoring device to be used for stabilizing the spine and the methods pertaining thereto.

### BACKGROUND OF THE INVENTION

Stabilization or fusion of the spine can alleviate back pain in a patient by taking the pressure off of the nerves that are responsible for causing the pain. This can be accomplished by restoring the alignment of the spine or the space between the vertebrae (e.g. by either a discectomy or a laminectomy) and then stabilizing the spine. The stabilization can result in either partial mobilization or complete immobilization depending on the desired surgical outcome. Spine stabilization with partial mobilization can stabilize the problem vertebra thus alleviating the pain, whereas complete spinal immobilization allows bone to grow between the stabilized vertebra, thus fusing the spine and alleviating the pain.

The spine can be stabilized using stabilization screws, such as pedicle, transarticular, lateral mass and laminar screws, which require anchoring the screw into the vertebrae and connecting the screws with a rod which then provides support to the spine in the restored position. In this stabilization system, the stabilization screws do not fixate the spinal segment, but act as anchor points which can be connected with a rod.

In some patients, the vertebrae is not capable of supporting the spine stabilization system, which system requires the pressure to be located on the anchoring points due to poor bone quality. Poor bone quality can be the result of insufficient bone density or a damaged (i.e. fractured or broken) vertebra.

In some cases, these stabilization screws are at risk of failure due to either bony failure or hardware failure. Bony failure typically occurs in patients with poor bone quality where the bone breaks at the anchoring point, whereas hardware failure is reported in patients with good bone quality and can be the result of the stabilization screw cracking or breaking. Such failures result in patient discomfort, subsequent spinal surgeries, and in some cases, can result in a patient being wheelchair-bound or possibly even death.

Therefore, spine stabilization systems which can be used on patients with poor bone quality would be useful in the treatment of chronic back pain.

### BRIEF SUMMARY OF THE INVENTION

The present invention is directed to a spine stabilization device which provides a distribution of the pressure over the laminar portion of the vertebrae and does not require the pressure of the spine stabilization system to be on the anchoring portion of the device. The spine stabilization device disclosed herein can serve as a rescue device which allows the spine to be stabilized in a patent with insufficient bone quality and/or damaged vertebrae. In addition, the spine stabilization system disclosed herein can serve as a rescue device in the event of stabilization screw failure.

The spine stabilization device disclosed herein comprises an elongated shaft portion for anchoring the spine stabilization device into a vertebra; a head portion, wherein the head portion comprises a groove for affixing a stabilization rod; and at least one aperture for housing a sublaminar wire which aperture passes through either the elongated shaft portion or the head portion of the spine stabilization device and is substantially perpendicular to the axis of the anchoring device.

In some embodiments, the spine stabilization device disclosed herein comprises only a head portion, wherein the head portion comprises a groove for affixing a stabilization rod; and an aperture for housing a sublaminar wire which aperture passes through the spine stabilization device and is substantially perpendicular to the axis of the anchoring device. In such embodiments, the spine stabilization device can be used on patents whose vertebrae is not capable of receiving the shaft of a screw or tack. The spine stabilization device is anchored to the vertebrae by the sublaminar wire.

In the spine, the invention provides a spine stabilization system comprising at least two spine stabilization devices, wherein the spine stabilization devices are anchored to at least two vertebra; a sublaminar wire such that at least one spine stabilization device comprises a closed sublaminar wire loop which is threaded through the aperture of the spine stabilization device and encircles the laminar region of the vertebra; a stabilization rod affixed to the head portion of the spine stabilization device wherein the stabilization rod connects each of the stabilization devices; and a screw nut inserted into the groove on the head portion of the spine stabilization devices to secure the stabilization rod thus providing stabilization for the spine thus providing stabilization for the spine. In some embodiments, more than one spine stabilization device comprises a closed sublaminar wire loop in the spine stabilization system. The sublaminar wire loop distributes the pressure applied to the vertebrae by the anchoring portion of the spine stabilization screw.

The present invention provides a method for stabilizing the spine comprising anchoring at least one of the spine stabilization devices of the invention to consecutive or non-consecutive vertebrae; affixing a sublaminar wire to the vertebrae, wherein the wire is threaded through the aperture of the spine stabilization device and clamped to form a closed loop encircling the laminar region of the vertebrae; connecting the spine stabilization device to one or more additional spine stabilization devices by inserting a stabilization rod into the groove of the head portion of the spine stabilization devices; and capping the groove on the head portion of the spine stabilization devices to secure the stabilization rod using a screw nut thus stabilizing the spine.

This sublaminar wire loop threaded through the aperture and affixed to the spine stabilization device provides not only a rescue mechanism in the event of stabilization device failure, but also when used as a primary spine stabilization device, it provides a mechanism for pressure and/or stress distribution thus reducing the instances and risk of stabilization device failure. The stress distribution provided by the sublaminar wire loop can also allow the anchoring portion of the device to be small, such that the length of the anchoring shaft is less than the thickness of the bone in which it is anchored. This alleviates the risk of surgical complications such as damage to the spinal cord, damage to the arteries and damage to nerves.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIGS. 1A, 1B and 1C show a plan view of a spine stabilization device of the invention. FIG. 1A shows a spine stabilization device in the form of a screw having the aperture in the anchoring shaft portion. FIG. 1B shows a spine stabilization device in the form of a tack having the aperture in the anchoring shaft portion. The tack can be pointed or rounded on the distal end of the anchoring shaft. FIG. 1C shows a spine stabilization device having the aperture in the head portion.

FIG. 2 shows a plan view of a spine stabilization device of the invention having the aperture in the head portion.

FIG. 3 shows a plan view of a spine stabilization device of the invention having an aperture in the anchoring shaft portion and an aperture in the head portion.

FIG. 4 shows a plan view of a spine stabilization device of the invention having the aperture in the anchoring shaft portion with the screw nut in place.

FIGS. 5A and 5B show a plan view of a spine stabilization device of the invention having the screw nut in place and a wire threaded through the head portion. FIG. 5A shows a cylindrical wire through a circular aperture, and FIG. 5B shows a ribbon-shaped wire through an elliptical aperture.

FIGS. 6A and 6B show the spine stabilization system of the invention in a spine. In FIG. 6A the sublaminar wire is not clamped to form a loop, whereas in FIG. 6B the sublaminar wire is clamped to form a closed loop

### DETAILED DESCRIPTION OF THE INVENTION

Before the present compositions and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

It must be noted that as used herein and in the appended claims, the singular forms “a”, “an”, and “the” include plural referents unless the context clearly dictates otherwise. Thus, for example, reference to “spine stabilization screw” includes a plurality of various spine stabilization screws and equivalents thereof known to those skilled in the art.

### 1. Definitions

Unless defined otherwise, all technical and scientific terms used herein have the same meaning as commonly understood by one of ordinary skill in the art to which this invention belongs. As used herein the following terms have the following meanings.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

The term “about” when used before a numerical designation, e.g., temperature, time, amount, and concentration, including range, indicates approximations which may vary by (+) or (−) 10%, 5% or 1%.

The term “aperture” is intended to refer to a hole or bore which passes through a solid portion of the stabilization device and is substantially perpendicular to the axis of the anchoring device. This allows the sublaminar wire loop to lie against the bone. The aperture can be created in the device as a post production modification or as part of the creation (e.g. casting) process. The aperture can have any cross-sectional shape, such as cylindrical, elliptical, D-shaped, triangular, rectangular, etc.

The term “biocompatable” is intended to refer to a is a synthetic or natural material which is non-toxic and elicits little or no immune response in a given organism when placed in intimate contact with living tissue. Biocompatible materials are intended to interface with biological systems to evaluate, treat, augment or replace any tissue, organ or function of the body and is intended to include biomimetic materials which are materials not made by living organisms but have compositions and properties similar to those made by living organisms. For example, calcium hydroxylapatite which is a coating on many artificial bones used as a bone replacement and allows for easier attachment of the implant to the living bone.

The term “D-shaped” is intended to refer to an aperture wherein the cross-section is D-shaped or semi-circular, i.e. the aperture has one flat side and one rounded side.

The terms “ribbon-shaped” and “ribbon-like” are intended to refer to a wire wherein the thickness of the wire is less than about 50% of the width of the wire. In some embodiments, the cross-section is rectangular. In some embodiments, the cross-section is elliptical.

The term “groove” is intended to refer to a gap or channel in the head portion of the stabilization device for housing the spine stabilization rod. The groove can be cut out of the head portion in at any orientation which allows insertion of the stabilization rod. For example, the groove can be in the top of the head portion as shown in the figures, or in the side of the head portion.

The term “rotatably connected” is intended to refer to a connectivity between the elongated shaft portion and the head portion of the device that allows the head portion to rotate or revolve around the axis defined by the shaft portion, which is fixed into the bone. Such connectivity includes, but is not limited to, a ball and socket connection.

### 2. Device of the Invention

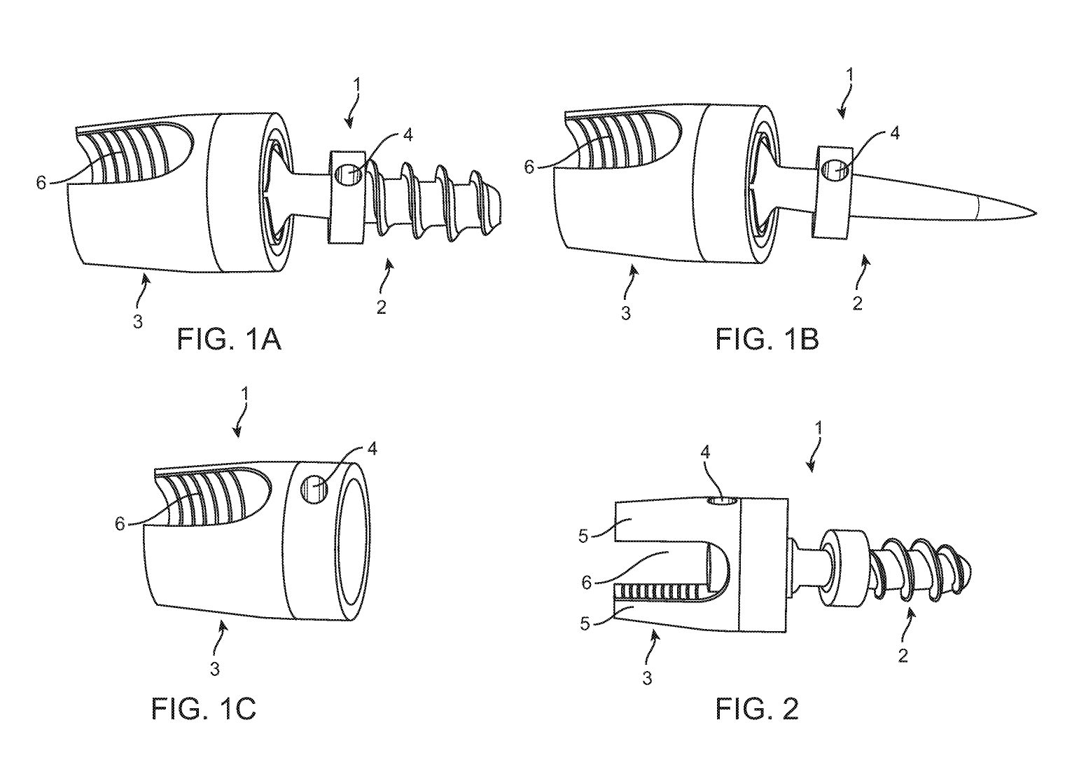

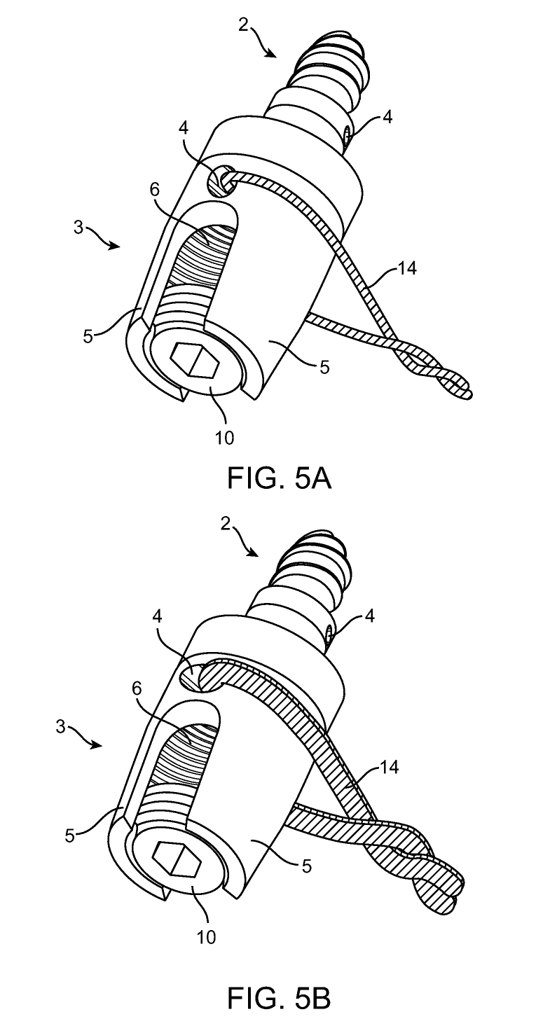

As shown in FIGS. 1A, 1B and 2, in one embodiment, the spine stabilization device 1 comprises an elongated shaft portion 2 for anchoring the spine stabilization device into a vertebra; a head portion 3, wherein the head portion comprises a groove 6 for affixing a stabilization rod 16; and at least one aperture 4 for housing a sublaminar wire 14 (FIGS. 5A and 5B) which aperture 4 passes through either the elongated shaft portion 2 (FIGS. 1A and 1B) or the head portion 3 (FIGS. 1C and 2) of the spine stabilization device 1 and is substantially perpendicular to the axis of the anchoring device.

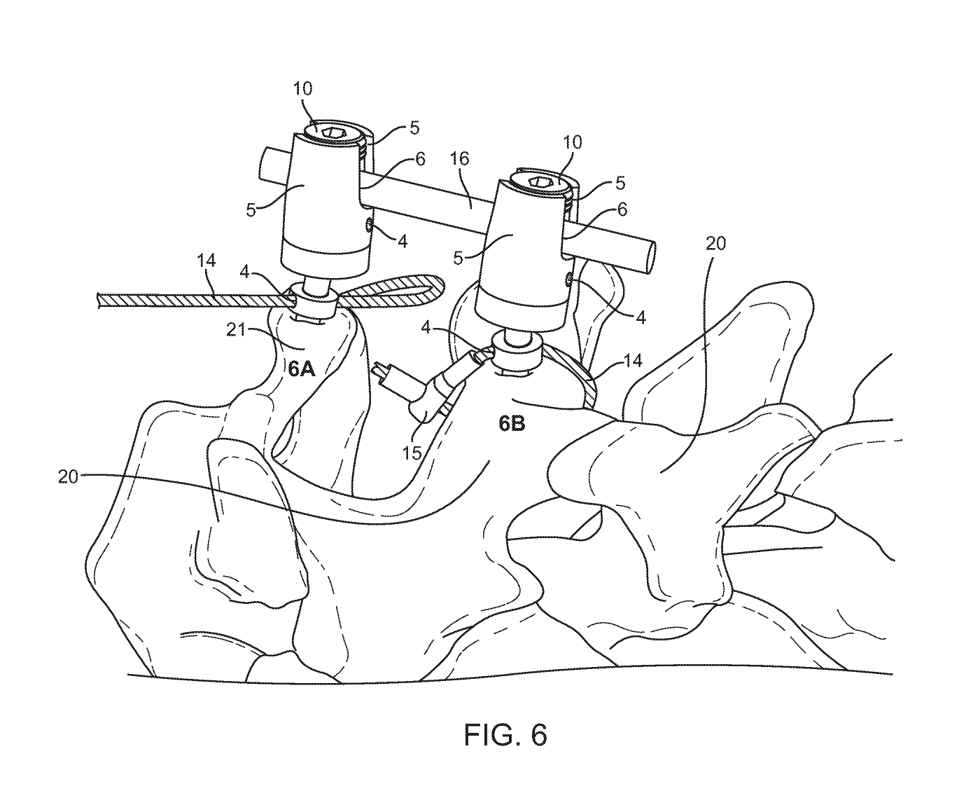

The anchoring shaft portion 2 of the anchoring device 1 is intended to be inserted into the bone of the spine 20 (FIG. 6). Accordingly, the anchoring shaft portion 2 can be smooth (such as in a nail or a tack, FIG. 1B) or may contain threads (such as in a screw or a bolt, FIG. 1A).

The head portion 3 of the spine stabilization device 1 comprises a groove 6 for affixing a stabilization rod 16 (FIG. 6), which can either be on the top or the side of the head portion 3. The head portion 3 of the spine stabilization device can either be permanently attached to the elongated shaft portion 2 or can be attached after the shaft portion 2 has been anchored into the vertebrae 20. In addition, the head portion 3 may comprise multiple components which can be assembled either before or after the shaft portion 2 is anchored into the vertebra 20.

In some embodiments, as shown in FIG. 1C, the spine stabilization device 1 comprises only a head portion 3, wherein the head portion 3 comprises a groove 6 for affixing a stabilization rod 16; and an aperture 4 for housing a sublaminar wire 14. Such spine stabilization devices can be anchored to the vertebrae 20 by the sublaminar wire loop 14.

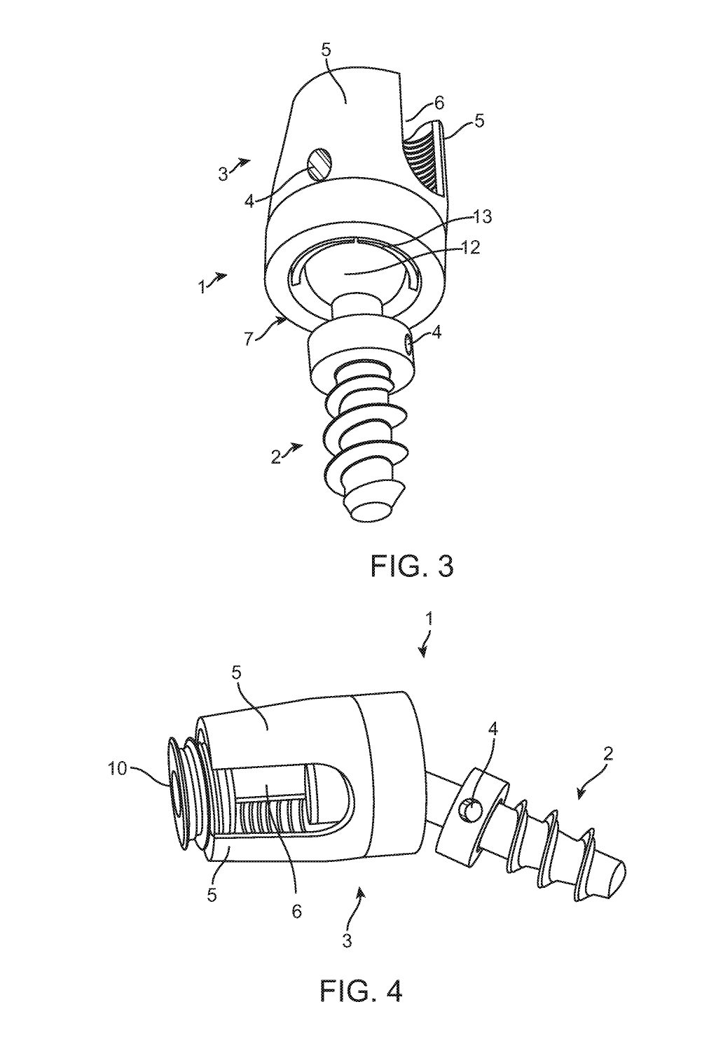

In some embodiments, the head portion 3 can be attached to the shaft portion 2 to allow movement or provide a fixed connection such that no movement of the head portion 3 is allowed (FIG. 4). When the head portion 3 is rotatably connected to the shaft portion 2 at a pivot point 7, this provides a polyaxial spine stabilization device. As shown in FIG. 3, the head portion can be connected to the shaft portion at a pivot point 7. This can be, for example, a ball and socket connection. FIG. 3 shows a ball 12 and socket 13 connectivity where the elongated shaft 2 comprises a spherical end 12 which is housed within a socket 13 of the head portion 3.

This polyaxiality allows the stabilization rod 16 to be affixed into the groove 6 of the head portion 3 without the need for presurgical alignment and extensive rod 16 manipulation which can decrease overall surgical time.

As shown in FIGS. 1A, 1B, 1C and 2, the spine stabilization device 1 comprises at least one aperture 4 for housing a sublaminar wire 14. The aperture 4 can pass through either the elongated shaft portion 2 or the head portion 3 of the spine stabilization device and is substantially perpendicular to the axis of the anchoring device so that the sublaminar wire 14 can suitably secure the spine stabilization device to the vertebra 20. In the case where the aperture 4 passes through the head portion 3 of the device, it should not be located within the groove 6, but within a solid portion of the base of the head portion 3 such that the wire 14 has sufficient structural integrity. However, when the aperture 4 passes through the head portion 3 of the spine stabilization device, any polyaxiality of the device is forfeited upon fixation with the sublaminar wire 14.

The spine stabilization device of the invention may be comprised of a biocompatible material, such as for example but not limited to, titanium, aluminum, gold, platinum, tantalum, niobium, iron, chromium, cobalt, magnesium, aluminum, palladium, vanadium, zirconium, chromium, nickel, molybdenum, stainless steel, and alloys thereof. In some embodiments, the material is magnetic resonance imaging (MRI) compatible (i.e. non-magnetic). In one embodiment, the material comprises titanium. The shaft portion and the head portion 3 can be made of any of the above materials and can be made of either the same or different material as the shaft 2 portion.

The cross-section of the aperture 4 can be any shape, such as circular, rectangular, elliptical, D-shaped or triangular to house sublaminar wires 14 of different shapes, such as cylindrical or ribbon-like wires (FIGS. 5A and 5B). It is contemplated that ribbon-like wires would provide additional stress distribution and therefore the aperture 4 may be either D-shaped, elliptical, or rectangular in such an instance.

As is shown in FIG. 6, provided herein is a spine stabilization system comprising at least one of the spine stabilization devices of the invention and, optionally, one or more spine stabilization devices, such as a pedicle screw, a lateral mass screw, a transarticular screw or a laminar screw. In some embodiments, the spine stabilization system comprises two or more of the spine stabilization devices of the invention.

In one embodiment of the spine stabilization system, the elongated shaft portion 2 of the spine stabilization devices are anchored into at least two vertebra 20. In some embodiments, the at least two vertebra 20 are not consecutive. In other embodiments, the at least two vertebra 20 are consecutive or adjacent to one another. The shaft portions can be anchored into the laminar 21 region of the vertebra 20. In one preferred embodiment, the shaft portion 2 is anchored into the lamina 21 such that the shaft portion 2 is substantially perpendicular to the lamina 21. As such, the shaft portion 2 of the device should be short enough such that it is less than the thickness of the lamina 21. This alleviates the risk of surgical complications such as damage to the spinal cord, arteries and nerves. Accordingly, in one embodiment, the shaft portion 2 of the device is no more than about 1 cm long and has a diameter of from about 2 mm to about 1 cm, or alternatively, from about 3 mm to about 8 mm, or alternatively, from about 4 mm to about 6 mm.

In such cases where the bone quality of the patient does not permit anchoring of a device into the vertebrae, one or more of the spine stabilization devices as shown in FIG. 1C can be used. Therefore, in some embodiments, the system comprises the spine stabilization device as shown in FIG. 1C, which comprises only a head portion 3, wherein the head portion 3 comprises a groove 6 for affixing a stabilization rod 16; and an aperture 4 for housing a sublaminar wire 14. Such spine stabilization devices can be secured to the vertebrae 20 by the sublaminar wire loop 14.

The vertebra 20 can be in any region, or over multiple regions, including the cervical, thoracic and the lumbar regions. While not shown, more than two devices can be used in the system on consecutive or non-consecutive vertebrae. It is contemplated that multilevel fusions can thus be performed. In another embodiment, the spine stabilization devices can be used in combination with other screw constructs, such as pedicle screws, lateral mass screws, transarticular, and/or laminar screws. It is contemplated that the spine stabilization system of the present invention can be used, either alone or in combination with other such constructs to stabilize the entire spine. In one embodiment, the vertebrae 20 are in the cervical region of the spine. In one embodiment, the vertebrae 20 are the C1 and C2 vertebrae 20.

The spine stabilization system further comprises at least one sublaminar wire (or cable) 14 such that at least one spine stabilization device comprises a sublaminar wire 14 threaded through the aperture 4 of the spine stabilization device. The sublaminar wire 14 must be capable of encircling the lamina and clamping on to itself to form a closed sublaminar wire 14 loop. The loop is formed by affixing one end of the sublaminar wire to the other end. The system provides security for the anchoring portion and prevents lateral movement of the sublaminar wire 14. In some embodiments, more than one spine stabilization device comprises a closed sublaminar wire 14 loop.

The sublaminar wire 14 loop can be formed by affixing one end of the sublaminar wire to the other end closed by any suitable means, such as tying, twisting or crimping the sublaminar wire 14. Other suitable means for affixing the sublaminar wire 14 include overlapping the ends of the wire 14 and clamping the ends together with a bracelet or with screws to form the loop. Any known means for affixing the sublaminar wire 14 into a loop can be used. Such methods for forming a closed sublaminar wire 14 loop are well known in the art. As is shown in FIG. 6B, the loop 14 can be formed with a crimper 15 using a standard handheld crimping tool known in the art.

The thickness or diameter of the sublaminar wire 14 should be less than that of the internal diameter of the aperture 4 in the stabilization device to allow for easy insertion by the clinician during surgery, but thick enough such that it can support the spine stabilization system. Sublaminar wires are commercially available in a variety of biocompatible materials, cross-sectional shapes (i.e. cylindrical or ribbon-shaped) and in a range of thicknesses. It is contemplated that any wire can be used provided that it has a sufficient tensile strength to secure the spine stabilization system to the vertebra and can be threaded through the aperture 4 of the system disclosed herein. It is contemplated that suitable wires 14 are from about 0.2 mm to about 3 mm, or alternatively, from about 0.5 mm to about 2 mm, or alternatively, from about 0.5 mm to about 1 mm. Therefore, in certain embodiments, the diameter of the aperture 4 (or each dimension of a non-spherical aperture 4) should be from about 0.3 mm to about 4 mm, or alternatively, from about 0.5 mm to about 3 mm, or alternatively, from about 0.8 mm to about 1.2 mm. In some embodiments the aperture 4 is spherical and has an internal diameter of from about 0.8 mm to about 2 mm.

The sublaminar wire 14 can be made of any biocompatible material that can provide the necessary structural integrity in the event of failure. Suitable materials include metals or metal alloys, such as titanium, a titanium alloy, platinum, a platinum alloy (e.g., a platinum-tungsten alloy), stainless steel and combinations thereof. In some embodiments, the material is magnetic resonance imaging (MRI) compatible. In one embodiment, the material comprises titanium. In some embodiments, the wire is coated with a biocompatible material. For example, the wire may be coated with a biocompatible polymer coating which is capable of eluting a therapeutic agent, such as an antibiotic, an anti-inflammatory agent, etc., to the surgical site. Such biocompatible polymer coatings for drug delivery are well known in the art and are available from commercial sources (Vertellus Specialties UK Ltd, United Kingdom).

In some embodiments, the wire 14 further comprises a means for stabilizing the wire 14 to the vertebrae 20. In some embodiments, the wire 14 comprises a biocompatible substance (i.e. a biocompatible rubber) or is textured with grooves or protrusions, for example, on at least a portion of the wire 14 such that the wire 14 is capable of gripping the vertebrae 20 thus stabilizing the device. In one embodiment, the wire 14 comprises grooves on at least a portion of the wire such that the wire 14 is stabilized to the vertebrae 20.

In some embodiments, the elements of the spine stabilization system, such as each independently the head portion 3, the shaft portion 2, and the sublaminar wire 14, comprise a biocompatible coating. Suitable biocompatible coatings can include therapeutic agents such as antibiotics or anti-inflammatory agents, or the components can have a coating to increase the biocompatibility, such as hydroxyapatite which mimics poor bone quality.

To provide the spine stabilization, a stabilization rod 16 is affixed to the head portion 3 of the spine stabilization devices, thus connecting each of the stabilization devices. The rod 16 affixed to the head portion 3 of the spine stabilization device by capping the groove 6 with a screw nut 10. The screw nut 10 is threadbly received by the prongs 5 on the head portion 3 that make up the groove 6 (FIG. 4).

The thickness or diameter of the stabilization rod 16 should be less han that of the width and depth of the groove 6 on the head portion 3 to allow for easy insertion by the clinician during surgery, but thick enough to provide suitable support to the spine. Accordingly, in one embodiment, the stabilization rod 16 has a diameter of from about 1 mm to about 1 cm, or alternatively, from about 2 mm to about 8 mm, or alternatively, from about 3 mm to about 6 mm. The stabilization rod 16 can be made of any biocompatible material that can provide the necessary support to the spine. Suitable materials include metals or metal alloys, such as platinum, a platinum alloy (e.g., a platinum-tungsten alloy), stainless steel and combinations thereof.

### 3. Methods of the Invention

Provided herein are methods for stabilizing the spine. The methods disclosed herein can comprise one or more of the spine stabilization devices as disclosed herein, alone or in combination with, other spine stabilization screws, such as pedicle, transarticular, lateral mass and laminar screws.

In one embodiment, the method of the invention comprises anchoring at least one of the spine stabilization devices of the invention to consecutive or non-consecutive vertebrae; affixing a sublaminar wire 14 to the vertebrae 20, wherein the wire is threaded through the aperture 4 of the spine stabilization device and clamped to form a closed loop encircling the laminar region of the vertebrae 20; connecting the spine stabilization device to one or more additional spine stabilization devices by inserting a stabilization rod 16 into the groove 6 of the head portion 3 of the spine stabilization devices; and capping the groove on the head portion of the spine stabilization devices to secure the stabilization rod using a screw nut thus stabilizing the spine.

In one embodiment, the one or more additional spine stabilization devices comprises a spine stabilization device as disclosed herein. In one embodiment, the one or more additional spine stabilization devices comprise one or more of a pedicle screw, a lateral mass screw, a transarticular screw or a laminar screw. In some embodiments, the method comprises two or more of the spine stabilization devices of the invention.

In such cases where the bone quality of the patient does not permit anchoring of a device into the vertebrae, one or more of the spine stabilization devices as shown in FIG. 1C can be used. Therefore, in some embodiments, the method of the invention comprises attaching to the vertebra 20 at least one of the spine stabilization devices of the invention by affixing a sublaminar wire 14 to the vertebrae 20, wherein the wire 14 is threaded through the aperture 4 of the spine stabilization device and clamped to form a loop; connecting the spine stabilization device to one or more additional spine stabilization devices by inserting a stabilization rod 16 into the groove 6 of the head portion 3 of the spine stabilization devices and capping the groove on the head portion of the spine stabilization devices to secure the stabilization rod using a screw nut thus stabilizing the spine.

In one embodiment, the method comprises at least one spine stabilization device as disclosed herein in combination with one or more of a pedicle screw, a lateral mass screw, a transarticular screw or a laminar screw. In some embodiments, the spine stabilization system comprises two or more of the spine stabilization devices of the invention.

The rod 16 can then be further secured into the groove 6 by capping the groove 6 on the head portion 3 of the spine stabilization devices using a screw nut 10. In some embodiments, the at least two vertebra 20 are not consecutive. In other embodiments, the at least two vertebra 20 are consecutive or adjacent vertebra 20.

Methods for anchoring spine stabilization devices such as screws and tacks into the spine are well known and practiced in the art, and generally require a drill (e.g., in the case of a screw or bolt) or mallet (e.g., in the case of a tack or nail) for anchoring the stabilization device into the vertebra. A drill may be used to remove the outer cortex of the bone and allow for insertion of the shaft portion 2 of the stabilization device. Similar methods can be used to anchor the shaft portion of the spine stabilization devices of the present invention.

The spine stabilization devices of the invention can be anchored into any region of the vertebra 20. In one embodiment, the spine stabilization devices are anchored into the laminar 21 region of the vertebra 20. Care should be taken in the selection of the anchoring device, as the shaft portions of the devices should be less than the thickness of the bone in which they are anchored. This alleviates the risk of surgical complications such as damage to the spinal cord, arteries and nerves. In one embodiment, the method comprises anchoring the devices into the laminar 21 region of the vertebra 20.

The consecutive vertebra 20 in which the devices are anchored can be in any region of the spine that would require stabilization, including the cervical, thoracic and the lumbar regions.

In one embodiment, the consecutive vertebrae 20 are in the cervical region of the spine. In one embodiment, the consecutive vertebrae 20 are the C1 and C2 vertebrae 20.

Spinal surgeries are typically performed in an open surgery which requires a 6 to 12 inch incision into the back of the patient. Although in some cases this type of surgery may not be avoidable, it is preferred that a smaller incision is made. By utilizing tubular retractor systems, for example, which are up to 26 millimeters in diameter, an incision of up to three inches may be required. It is contemplated that the methods and devices disclosed herein provide for a minimally invasive alternative to traditional methods for spine surgeries. It is contemplated that the methods disclosed herein can be performed using micro incisions of about three inches or less. In some embodiments, the incision is less than about 2 inches, and in some embodiment, the incision is less than about 1 inch.

It will be appreciated that those skilled in the art will be able to devise various arrangements which, although not explicitly described or shown herein, embody the principles of the invention and are included within its spirit and scope. Furthermore, all conditional language recited herein is principally intended to aid the reader in understanding the principles of the invention and the concepts contributed by the inventors to furthering the art, and are to be construed as being without limitation to such specifically recited conditions. Moreover, all statements herein reciting principles, aspects, and embodiments of the invention are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A spine stabilization device comprising
a head portion, wherein the head portion comprises a groove for affixing a stabilization rod; and
at least one aperture for housing a sublaminar wire which aperture passes through the spine stabilization device and is substantially perpendicular to the axis of the anchoring device.

2. The spine stabilization device of claim 1, further comprising an elongated shaft portion for anchoring the spine stabilization device into a vertebra.

3. The spine stabilization device of claim 2, wherein the aperture passes through the elongated shaft portion of the anchoring device.

4. The spine stabilization device of claim 1, wherein the aperture passes through the head portion of the anchoring device.

5. The spine stabilization device of claim 1, wherein the cross-sectional shape of the aperture is circular, rectangular, elliptical or triangular.

6. The spine stabilization device of claim 2, wherein the spine stabilization device is a polyaxial spine stabilization device such that the elongated shaft portion is rotatably connected to the head portion.

7. The spine stabilization device of claim 1, wherein the interior of the groove in the head portion comprises threads for threadably receiving a screw nut.

8. The spine stabilization device of claim 2, wherein the elongated shaft portion comprises threads.

9. The spine stabilization device of claim 1, further comprising a sublaminar wire passing through an aperture of the spine stabilization device.

10. The spine stabilization device of claim 1, further comprising a sublaminar wire passing through an aperture of the spine stabilization device, wherein the sublaminar wire forms a closed loop.

11. The spine stabilization device of claim 10, wherein the sublaminar wire is cylindrical.

12. The spine stabilization device of claim 10, wherein the sublaminar wire is ribbon-shaped.

13. The spine stabilization device of claim 2, wherein the elongated shaft portion and the head portion are each independently comprised of a metal selected from the group consisting of titanium, aluminum, gold, platinum, tantalum, niobium, iron, chromium, cobalt, magnesium, aluminum, palladium, vanadium, zirconium, chromium, nickel, molybdenum, stainless steel, and alloys thereof.

14. A spine stabilization system comprising
at least two spine stabilization devices wherein at least one is the device of any one of the above claims, and further wherein the spine stabilization devices are anchored to at least two vertebra;
at least one sublaminar wire such that at least one spine stabilization device comprises a sublaminar wire loop which is threaded through the aperture of the spine stabilization device and encircles the laminar region of the vertebrae;
a stabilization rod affixed to the head portion of the spine stabilization devices wherein the stabilization rod connects each of the stabilization devices; and
a screw nut inserted into the groove on the head portion of the spine stabilization devices to secure the stabilization rod thus providing stabilization for the spine.

15. The spine stabilization system of claim 14, wherein the vertebra are consecutive.

16. The spine stabilization system of claim 14, further comprising more than one sublaminar wire such that more than one spine stabilization device comprises a sublaminar wire loop which is threaded through the aperture of the spine stabilization device and encircles the laminar region of the vertebra.

17. The spine stabilization system of claim 14, wherein the groove on the head portion of the spine stabilization devices is capped by a screw nut.

18. The spine stabilization system of claim 14, wherein the vertebra are in the cervical region of the spine.

19. The spine stabilization system of claim 18, wherein the vertebra are the C1 and C2 vertebrae.

20. The spine stabilization system of claim 14, wherein the vertebra are in the thoracic region of the spine.

21. The spine stabilization system of claim 14, wherein the vertebra are in the lumbar region of the spine.

22. The spine stabilization system of claim 14, wherein the spine stabilization devices are anchored to consecutive vertebra.

23. The spine stabilization system of claim 14, wherein the spine stabilization devices are anchored to the laminar region of the vertebra.

24. A method for stabilizing the spine comprising
anchoring at least one of the spine stabilization devices of claim 1 to consecutive or non-consecutive vertebrae;
affixing a sublaminar wire to the vertebrae, wherein the wire is threaded through the aperture of the spine stabilization device and clamped to form a closed loop encircling the laminar region of the vertebrae;
connecting the spine stabilization device to one or more additional spine stabilization devices by inserting a stabilization rod into the groove of the head portion of the spine stabilization devices; and
capping the groove on the head portion of the spine stabilization devices to secure the stabilization rod using a screw nut thus stabilizing the spine.

25. The method of claim 24, wherein the sublaminar wire loop is clamped with a crimper.

26. The method of claim 24, wherein the vertebra are consecutive.

27. The method of claim 26, wherein the consecutive vertebra are in the cervical region of the spine.

28. The method of claim 27, wherein the consecutive vertebra are the C1 and C2 vertebrae.

29. The method of claim 26, wherein the consecutive vertebra are in the thoracic region of the spine.

30. The method of claim 26, wherein the consecutive vertebra are in the lumbar region of the spine.

31. The method of claim 24, wherein the spine stabilization devices are anchored to the laminar region of the vertebra.

32. The method of claim 24, wherein the method is performed using a minimally invasive procedure.

33. The method of claim 32, wherein the minimally invasive procedure comprises an incision of about 3 inches or less.

34. The method of claim 32, wherein the minimally invasive procedure comprises an incision of about 1 inch or less.
